(19) United States
(12) Patent Application Publication
(10) Pub. No.: US 2004/0187873 A1
(43) Pub. Date: Sep. 30, 2004

(54) Human jaw supportive device for reducing snoring and obstructive sleep apnea

(76) Inventor: C. Stephen Brown, Milton, FL (US)

Correspondence Address: C. Stephen Brown, P.O. Box 11214, Pensacola, FL, 32524, US

(21) Appl. No.: 10/401,441

(22) Filed: Mar. 28, 2003

(51) Int. Cl. A61F 5/56; A61F 11/00

(52) U.S. Cl. 128/848; 128/857

(57) ABSTRACT

A human jaw supportive device consisting essentially of elastic textile products. The supporter has members that encircle the head and apply upward pressure on the lower jaw and the bottom of the chin to stabilize them while the user is sleeping and to keep the jaw in a desired position while sleeping. The supporter is a device for preventing snoring and obstructive sleep apnea.

When the supporter's elastic members are placed over the top of the user's head with the elastic member under the user's chin, the supporter keeps the users mouth closed so that air flows through the nasal passages. The supporter stabilizes the lower jaw and prevents the lower jaw from overextending toward the user's neck, and prevents the tongue from falling back and blocking the user's airway and breathing passage.

### BACKGROUND OF THE INVENTION

In general, the present invention relates to devices for treating sleeping problems, particularly some forms of obstructive sleep apnea and snoring.

Many jaw supportive devices have been designed to keep a persons mouth closed, while sleeping, in an effort to prevent snoring and obstructive sleep apnea.

People, who mouth breathe while they are sleeping, usually begin at the age of 5 years old. Left untreated, often the muscles and tissue in the jaw lengthen further than they were intended causing snoring and obstructive sleep apnea. Mouth breathing also causes unnecessary ingestion of airborne germs, dust, other particles and drooling.

Prior methods of treating mouth breathing, snoring and sleep apnea are disclosed in U.S. Pat. Nos. 5,893,365; 6,279,577; 5,361,416; D331, 155; 5,499,633; and 5,687,743. The background of the invention of U.S. Pat. No. 5,893,365 contains a detailed description of the conditions that predicate the problem of snoring and obstructive sleep apnea and methods and devices that mitigate and control snoring and sleep apnea.

When the nasal passages are closed, the user breathes through the mouth. The nose airway passages remain congested while the interior of the mouth, tongue and lips become dry. Mouth breathing bypasses the natural filtration in the nose. The supporter applies pressure on the chin which retains the user's mouth closed so that air flows through the nasal airway passages. The natural structure of the nose filters the air to remove foreign particles that can harm lung tissues. The air flowing in the nasal airway passages also relieves nasal congestion and improves the quality of sleep. The supporter when placed about a user's head relaxes the bones, joints and muscles of the person's face to alleviate temporomandibular joint dysfunction.

The supporter includes a comfortable one-piece polyester and elastic design that fits around the face and chin of a human to overcome breathing and snoring problems. The supporter keeps the mouth closed during the sleep state when snoring commonly occurs. While awake, the supporter allows the user to breathe through the mouth talk eat, drink or cough. The elasticity of the supporter, and the various different sizes that can be made, allows a user, with any head circumference and any shape of chin to use it comfortably. The supporter has no projecting structures on any side of the head that can cause the user discomfort while sleeping. Unlike prior methods, the present invention tends to stay on the head of the user while sleeping because the front of the jaw supporter pass on either side of the ears. The jaw supporter is made of fabrics and materials that stretch and conform to the users unique head shape and size.

The front member touches, or comes very close to, the bottom edge of the user's lower lip. The front member applies an upward force on the bottom of the lip to seal the lips and reduce snoring noise, dry mouth, and nasal congestion, the unnecessary ingestion of foreign airborne particles that can harm lung tissues, and cause the user to breathe through the nasal passageways. The natural structure of the nose filters the air to remove foreign particles.

The front member applies an upward force on the bottom of the chin to lift the tongue from the posterior pharyngeal wall to open the airway passage and retain the passage open.

The rear member applies an upward force on the bottom of the chin to lift the tongue from the posterior pharyngeal wall to open the airway passage and retain the passage open.

### BRIEF SUMMARY OF THE INVENTION

The device of the invention is a supporter of the human lower jaw and is designed to satisfy the aforementioned needs for reducing snoring and obstructive sleep apnea activities.

The jaw supporter of the present invention is a comfortable, inexpensive and effective jaw supportive device.

A recent prior device disclosed by Isabelle Goodwin in U.S. Pat. No. 5,687,743 is a lower jaw supporter that utilizes adjustable straps. The adjustable straps overlap each other and create unnecessary pressure and discomfort to the users face and head in overlapping areas. Also, the chin area is left uncovered by the device, which may reduce the structural integrity and comfort of the device. The Goodwin device may be effective in use, however, it does not provide the most comfortable solution for the problems at hand.

### DETAILED DESCRIPTION OF THE INVENTION

Referring to FIGS. 1 to 7, there is illustrated a human lower jaw supporter, designated 10, of the present invention for applying upward force to the bottom of a user person's chin and lower jaw to support and stabilize the lower jaw while sleeping. The supporter 10 prevents the lower jaw from moving back toward the neck and prevents the tongue from being able to move back and close off the person's air passage. Supporter 10 retains the person's mouth closed, inhibits the closing of the nasal airway passages, and mitigates sleep apnea and snoring. Mouth breathing is not restricted.

Supporter 10 is made from two pieces of textile fabrics 22 and 23, of which can be 100% polyester or other suitable textile materials, or a combination of different kinds of textile materials. The present invention is a two ply supporter but may be comprised from a single ply of a suitable textile product or other elastic material or fabric. Both pieces of textile materials 22 and 23 are cut 26 inches long using identical patterns and the patterns can vary in length to obtain a suitable, different size. The two separate pieces of textile fabrics 22 and 23 are laid directly on top of each other and sewn together using and over edge stitch, or other suitable stitch, at the edges 16 and 17. Opposing ends 24 and 25 of the jaw supporter are then sewn together to create a non-ending circumference shaped supporter. The circumference can vary in size to obtain suitable sizes for different users.

In FIG. 3 the front member 11 is 1.25 inches wide, but can have any other suitable width, and is attached to the rear member 12 by two 4 inch long elastic, or any other suitable material, link straps 13. The elastic link strap has a width of 0.5 inches and a length of 4 inches, but can have any other suitable width or length. The rear member 12 is 1.25 inches wide, but can have any other suitable width, and is attached to the front member 11 by the opposing ends of the two elastic link straps 13 attached to the front member 11.

In FIGS. 1, 2, 3, 4, and 7, a border 14 and 15 made of polyester, or any other suitable material, with a 3/16 inch wide, or any other suitable width or thickness, strand of elastic encased inside the borders 14 and 15 is stitched onto the front and rear members 11 and 12 along the outer edges 27 and 28 of the supporter. The borders 14 and 15 can have any other suitable size of elastic, or any other suitable type of material that stretches and retracts, including but not limited to, gum rubber, encased inside the borders 14 and 15.

The jaw supporter is to be worn on the human head by placing the part of the supporter E that covers the human chin, which has a general width of 3 inches but can have any other suitable size, under the chin so that the front member border 14 is touching the bottom lip of the user, or as close as the user desires, while the rear member border 15 wraps under the chin, of the user, and extends towards the throat of the user.

The rear member 12 is placed over the top of the users head B and is positioned behind the persons ear C and rear member 12 is positioned down the side of the users face. The front member 11 shall remain forward of the users ear C and is positioned down the side of the users face.

The front member border 14 shall touch the bottom edge of the users lower lip D, or as close as the user desires, and applies an upward force on the bottom of the lip D to seal the lips and reduce snoring noise, dry mouth, nasal passage congestion, the unnecessary ingestion of foreign airborne particles that can harm lung tissues, and cause the user to breathe through the nasal passageway. The natural structure of the nose filters the air to remove foreign particles.

The front member border 14 also applies an upward force on the bottom of the chin to lift the tongue from the posterior pharyngeal wall to open the airway passage and retain the passage open.

The rear member border 15 applies an upward force on the bottom of the jaw to lift the tongue from the posterior pharyngeal wall to open the airway passage and retain the passage open.

The jaw supporter is not intended as a cure for obstructive sleep apnea and the user should always seek the medical advice of a physician trained specifically in the field of obstructive sleep apnea before relying on, or using, this or any other jaw supportive device. The jaw supporter should not be used if the user cannot breathe through their nasal passages.

### BRIEF DESCRIPTION OF THE DRAWINGS

FIG. 1 is a side view of a person wearing the jaw supporter;

FIG. 2 is a perspective side view of the jaw supporter;

FIG. 3 is a perspective top view of the jaw supporter;

FIG. 4 is a perspective bottom view of the jaw supporter;

FIG. 5 is a view of the first ply of fabric of the jaw supporter after is has been cut out from its pattern.

FIG. 6 is a view of the second ply of fabric of the jaw supporter after is has been cut out from its pattern;

FIG. 7 is a side view of the jaw supporter.

FIG. 8 is a perspective view of the border and the elastic encased within.

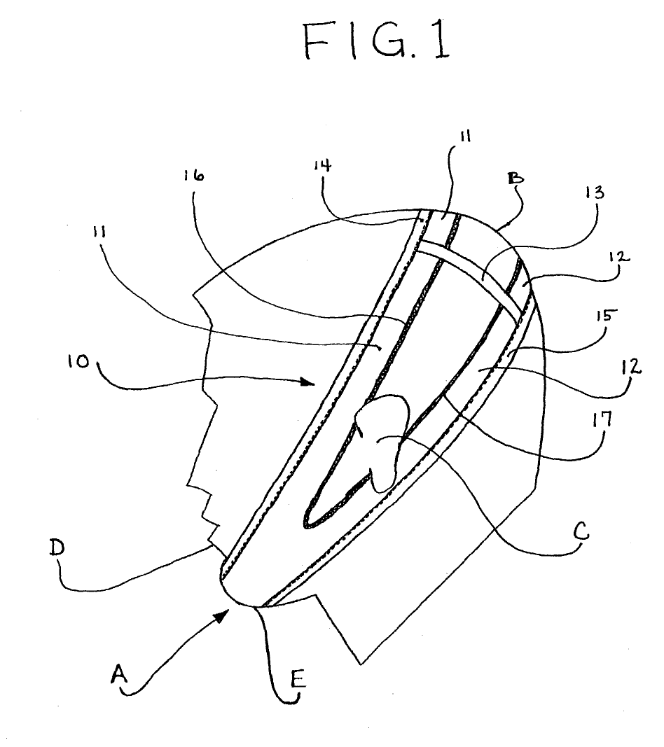

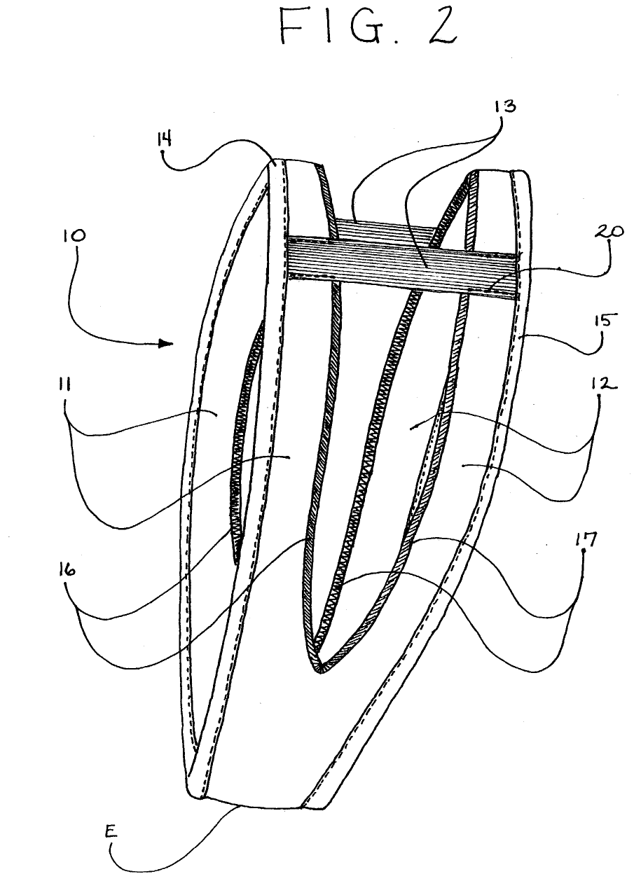

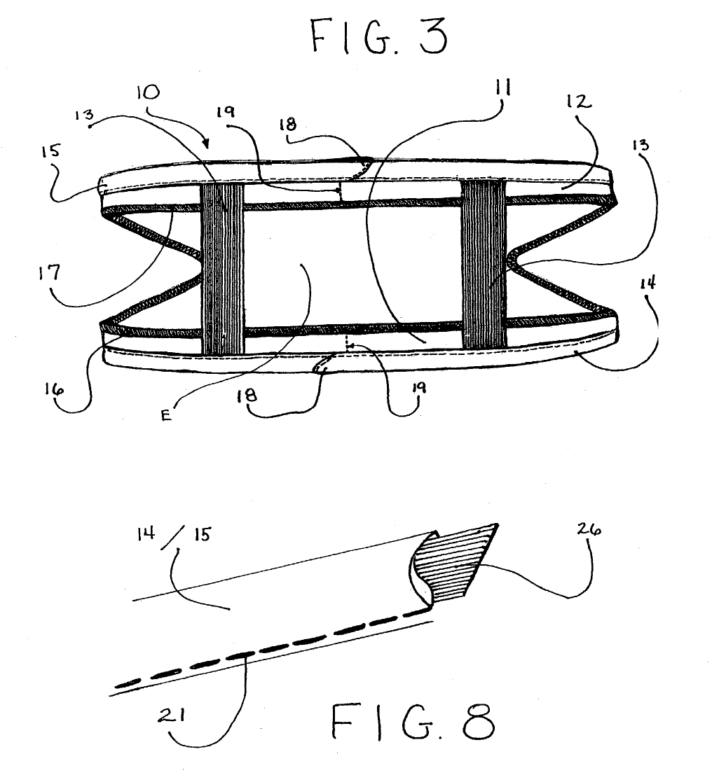

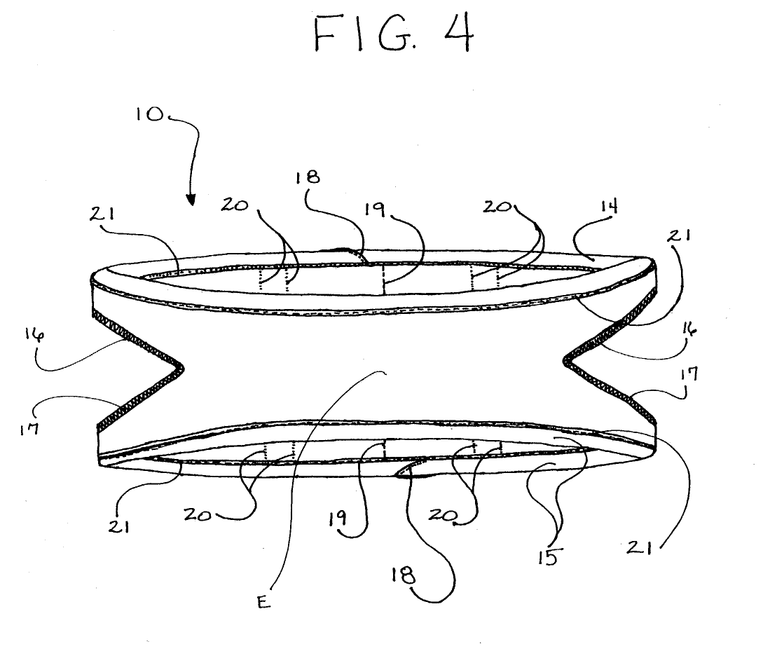

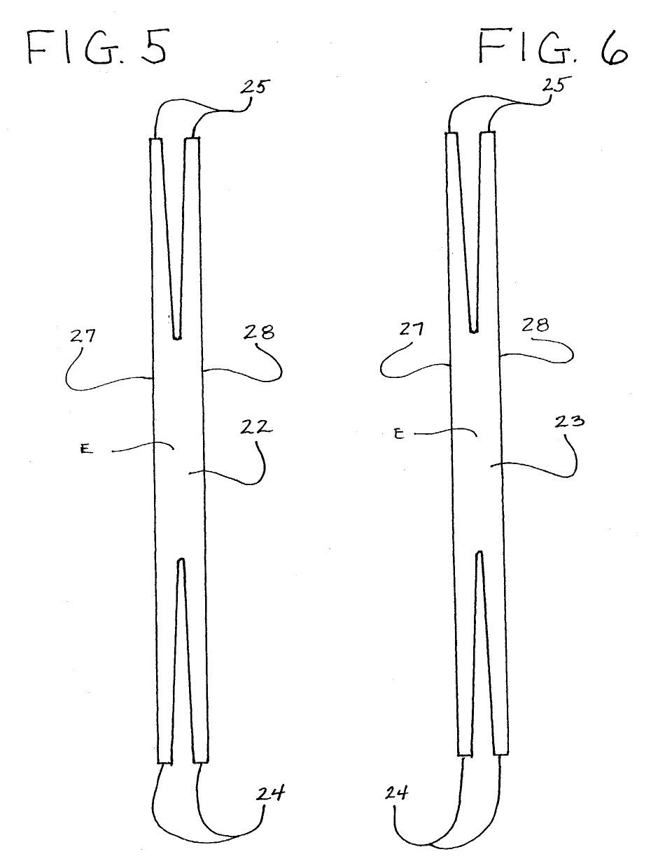

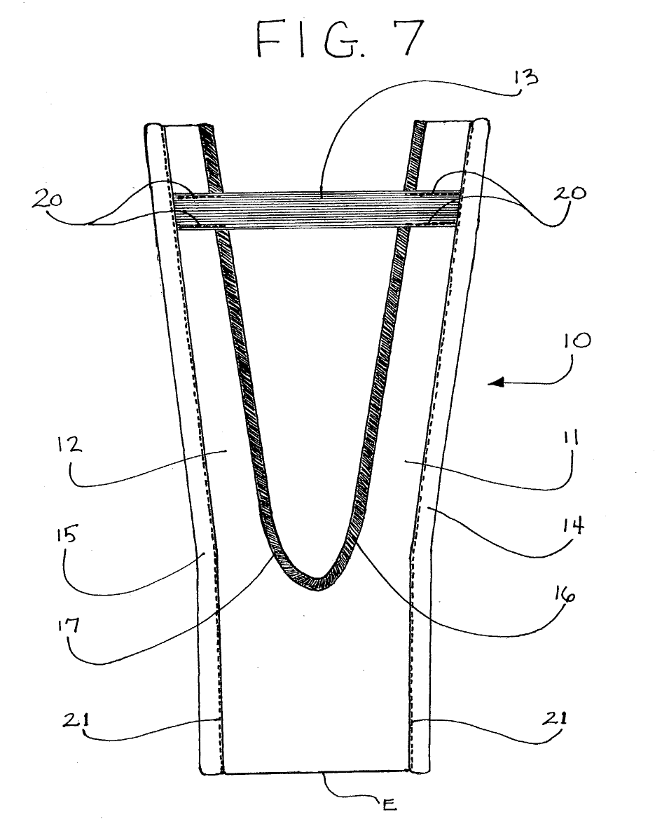

The object of the present invention is to effectively support the lower jaw while sleeping and provide the user with an extremely comfortable and desirable jaw supportive device that improves, encourages and enhances high quality sleep time.

Since the invention presented is made in different size circumferences from fabrics that stretch and retract, it easily adjusts to the shape and size of any head without the use of any type of fasteners.

The fabrics in the invention are made of materials that stretch and retract namely Spandex or other similar polyester fibers. Therefore, the invention can be used by a person indefinitely because it does not lose its elasticity, like other jaw supportive devices composed of fabrics that have lesser degrees of stretch memory. Additionally, the strength of the borders is fortified by an elastic band, encased therein, and further helps to prevent the supporter from losing its elasticity.

Both the front and rear border have a 3/16 wide strand of elastic inside of them. The elastic runs the full length of the circumference. The elastic can be installed into the border with or without being stitched into the border. However the elastic is stitched to the border, where the cut ends of the supporter have been sewn together. The encasement of elastic within the border is unique to the invention. The elastic is made of a grade and quality that provides recoil when it is not in use. Polyester elastic, gum rubber, or any other similar rubber, polyester, or elastic materials, can be used in the border for the purpose of providing the recoil of the border.

The supporter is designed to rely on the human ears, and the side of a person's head to keep the supporter in place while the person is using it.

The effectiveness of the supporter is also dependent on the width of the supporter, where the supporter wraps around the chin. In this area the supporter must be wide enough to touch the bottom lip of the person using it, and still wrap underneath the chin.

The front and rear borders of the supporter primarily work together to support the jaw and prevent it from moving towards the neck of the user while the user is sleeping. The front border places direct upward pressure at the bottom edge of the lower lip to keep the lips closed while sleeping.

I claim:

1. A jaw supportive device for reducing snoring and obstructive sleep apnea, said device comprising: an elastic and polyester means for holding the lower jaw in a selected position, said elastic and polyester means having front and rear members, of which the front member passes forward of the ears and the rear member passes behind the ears over the top of the head downwardly to below the ears where the front and rear members merge together to form a single member that passes under the chin of the user;

2. The supporter of claim 1 wherein; the forward and rear members are bound at their edges with polyester borders;

3. The supporter of claim 1 wherein; the polyester borders encase an elastic strand and are unique to the present invention;

4. The supporter of claim 1 wherein; the elastic strand in the polyester borders is contiguous through the entire border and are unique to the present invention;

5. The supporter of claim 1 comprises; an area of the supporter that makes contact with the chin, and said area of the supporter is sufficiently wide enough to make contact with the bottom edge of the users lower lip and extend downward and wrap under the chin and extend back towards the neck of the user;

6. The supporter of claim 1 comprises; an elastic link strap interconnecting said front and rear members and being spaced above the ears of the user and disposed on top of the head of the user, said link having front and rear opposite ends attached respectively to said front and rear members;

7. The supporter of claim 1 consisting essentially of; elastic materials that self adjust to the different shapes and sizes of the human head, face and chin;

8. The supporter of claim 1 is comprised; specifically to support and stabilize the lower jaw and keep the users mouth closed while the user is sleeping;

9. The supporter of claim 1 is comprised: to provide the user with a r comfortable jaw supporter that prevents the lower jaw from dropping further than its intended range while the user is sleeping.

10. The supporter of claim 1 is comprised of: lightweight fabrics that inhibit the build up of body heat underneath the materials or fabrics of the supporter.

11. The supporter of claim 1 is comprised of: Two plys of textile fabrics, similar in shape, placed directly on top of each other and sewn together at the edges.
